(19) United States
(12) Patent Application Publication
(10) Pub. No.: US 2024/0324860 A1
(43) Pub. Date: Oct. 3, 2024

(54) ENDOSCOPE

(71) Applicant: FUJIFILM Corporation, Tokyo (JP)

(72) Inventor: Takashi Harada, Kanagawa (JP)

(73) Assignee: FUJIFILM Corporation, Tokyo (JP)

(21) Appl. No.: 18/603,195

(22) Filed: Mar. 12, 2024

(30) Foreign Application Priority Data
Mar. 30, 2023 (JP) 2023-055355

(51) Int. Cl. A61B 1/005

(52) U.S. Cl. CPC A61B 1/0052; A61B 1/0057

(57) ABSTRACT

An endoscope includes: a rotation operating part that is rotatably provided on an endoscope operation unit and that moves a bending operation wire forward and backward by being rotationally operated; an engaging part that frictionally engages with the rotation operating part; and a pressing part that is movable between a braking position coming into contact with the engaging part and a non-braking position positioned on a distal side with respect to the engaging part from the braking position. The rotation operating part and the engaging part are integrally rotatable in a case where the pressing part is positioned at the non-braking position, and the rotation operating part and the engaging part are relatively rotatable in a case where the pressing part is positioned at the braking position.

### CROSS-REFERENCE TO RELATED APPLICATION

The present application claims priority under 35 U.S.C § 119(a) to Japanese Patent Application No. 2023-055355 filed on Mar. 30, 2023, which is hereby expressly incorporated by reference, in its entirety, into the present application.

### BACKGROUND OF THE INVENTION

### 1. Field of the Invention

The present invention relates to an endoscope.

### 2. Description of the Related Art

A flexible endoscope widely used for medical use generally includes an insertion part to be inserted into a body of a subject and an operating part gripped and operated by a practitioner. The insertion part is configured by, in order from a distal end side, being connected to a distal end hard part in which an objective optical system, a solid-state imaging element, or the like is built, a bendable part that is bent by a bending operation of an operating part, and a long soft part having flexibility.

The bendable part is configured such that a plurality of nodal rings are rotatably connected to each other to be bendable, a distal end part of an operation wire, which passes through an inside of the insertion part, is fixed to a leading nodal ring or distal end hard part, and a proximal end of the operation wire is connected to a bending operation mechanism of the operating part.

The operating part is provided with a bending operation knob operated by a practitioner as a component of the bending operation mechanism. By operating the bending operation knob, the operation wire is pushed and pulled, and the bendable part is bent in an up-down or left-right direction. As a result, the practitioner can operate the bending operation knob to change a bending state of the bendable part, and to direct the distal end hard part in a desired direction.

In the operation of the bending operation knob, even in a case where the practitioner releases his or her fingers from the bending operation knob, maintaining a state in which a bending state of the bendable part is changed may be desired such that the distal end hard part can be kept directed in a desired direction.

Therefore, in the related art, a mechanism for holding a bending posture of a bendable part is provided in an operating part of an endoscope. For example, WO2012-070321A, WO2020-070774A, and JP2021-137183A disclose a mechanism that applies a frictional force to the bending operation knob to fix (lock) the bending operation knob in order to maintain a state in which the bending state of the bendable part is changed.

### SUMMARY OF THE INVENTION

Meanwhile, in a case where the bending posture of the bendable part is held and the bending operation knob is completely fixed, the bending state of the bendable part by the bending operation knob cannot be finely adjusted. In this case, the practitioner needs to release a state in which the bending operation knob is completely fixed and to adjust the bending state of the bendable part again. Therefore, it is desirable that the frictional force with respect to the bending operation knob is at a magnitude in which the bending state of the bendable part can be maintained and finely adjusted by the operation of the fingers.

In a mechanism disclosed in WO2012-070321A, a friction plate is sandwiched between two plate-shaped fastening members to apply a frictional force to the bending operation knob. However, since the plate-shaped fastening member is unstable in a state in which the bending operation knob is not fixed (free state), there is a concern that the operability decreases due to occurrence of squeezing or abnormal noise and sudden increase in resistance during the operation caused by occurrence of backlash or distortion due to a direction of gravitational force, the operation of a practitioner, or the like.

In a mechanism disclosed in WO2020-070774A, light pressure is applied to a plate-shaped fastening member to prevent backlash in a free state of the bending operation knob, but resistance to the bending operation knob is increased, and the operation may be difficult to be performed by a practitioner.

In a mechanism disclosed in JP2021-137183A, a frictional force depends on a spreading amount between two pressing pieces. Since the spreading amount largely depends on finished dimensions of constituent members such as pressing pieces, there is a problem in that the frictional force is not stable and resistance of the bending operation knob varies depending on the product.

The present invention has been made in view of such circumstances, and an object of the present invention is to provide an endoscope that realizes stable operability by suppressing variations in a bending operation of the bendable part.

An endoscope according to an aspect of the present invention comprises a rotation operating part that is rotatably provided on an endoscope operation unit and that moves a bending operation wire forward and backward by being rotationally operated; an engaging part that frictionally engages with the rotation operating part; and a pressing part that is movable between a braking position coming into contact with the engaging part and a non-braking position positioned on a distal side with respect to the engaging part from the braking position, in which the rotation operating part and the engaging part are integrally rotatable in a case where the pressing part is positioned at the non-braking position, and the rotation operating part and the engaging part are relatively rotatable in a case where the pressing part is positioned at the braking position.

In an aspect of the present invention, the pressing part may be configured to be movable in a direction orthogonal to a rotary shaft of the rotation operating part.

In an aspect of the present invention, the non-braking position may be a position at which the pressing part is separated from the engaging part.

In an aspect of the present invention, the engaging part and the rotation operating part may be frictionally engaged with each other by a friction material.

In an aspect of the present invention, the friction material may be made of an elastic member.

In an aspect of the present invention, the endoscope further comprises a movement operation member that operates to move the pressing part between the non-braking position and the braking position.

In an aspect of the present invention, the endoscope further comprises a movable member that moves the pressing part between the non-braking position and the braking position in a case where the movement operation member is operated to move.

In an aspect of the present invention, the endoscope further comprises a rotating body that is rotatable around a rotary shaft of the rotation operating part in a case where the movement operation member is operated to move, in which the movable member is configured to be opened and closed between an open state and a closed state according to a rotational position of the rotating body, and the pressing part is positioned at the non-braking position in a case where the movable member is in the closed state, and the pressing part is positioned at the braking position in a case where the movable member is in the open state.

In an aspect of the present invention, the pressing part and the engaging part are frictionally engaged with each other in a case where the pressing part is positioned at the braking position.

In an aspect of the present invention, the pressing part and the engaging part are frictionally engaged with each other by a friction material.

In an aspect of the present invention, in a case where the pressing part is positioned at the braking position, a frictional force generated between the engaging part and the rotation operating part is defined as a first frictional force, and a frictional force generated between the pressing part and the engaging part is defined as a second frictional force, the first frictional force may be smaller than the second frictional force.

In an aspect of the present invention, the pressing part and the engaging part may be unevenly engaged with each other in a case where the pressing part is positioned at the braking position.

In an aspect of the present invention, the pressing part may be provided with a projection part, and the engaging part may be provided with a recessed part that engages with the projection part.

According to an aspect of the present invention, it is possible to realize stable operability by suppressing variations in a bending operation of a bendable part.

### BRIEF DESCRIPTION OF THE DRAWINGS

FIG. 1 is an overall configuration view of an endoscope according to an embodiment.

FIG. 2 is an enlarged perspective view of a main part of a distal end hard part as viewed from a distal end side.

FIG. 3 is a diagram for illustrating a bending operation mechanism.

FIG. 4 is a cross-sectional view taken along a rotary shaft of an operation knob according to a first embodiment.

FIG. 5 is a perspective view of an engaging part.

FIG. 6 is an assembly perspective view of a movable member.

FIG. 7 is a perspective view of the movable member of FIG. 6 as viewed from an opposite side.

FIG. 8 is a perspective view of an operation lever and a rotating body.

FIG. 9 is a diagram showing that the operation knob is in a free state as viewed from a direction of an arrow 9-9 in FIG. 4.

FIGS. 10A and 10B are diagrams showing that the operation knob is in a half-locked state as viewed from the same direction as in FIG. 9.

FIGS. 11A and 11B are diagrams for conceptually illustrating an action of a brake mechanism.

FIG. 12 is a cross-sectional view taken along a rotary shaft of an operation knob according to a second embodiment.

FIG. 13 is a diagram of a brake mechanism according to a third embodiment as viewed from a direction of a rotary shaft.

FIGS. 14A and 14B are diagrams for conceptually illustrating an action of a brake mechanism according to a fourth embodiment.

### DESCRIPTION OF THE PREFERRED EMBODIMENTS

### Endoscope

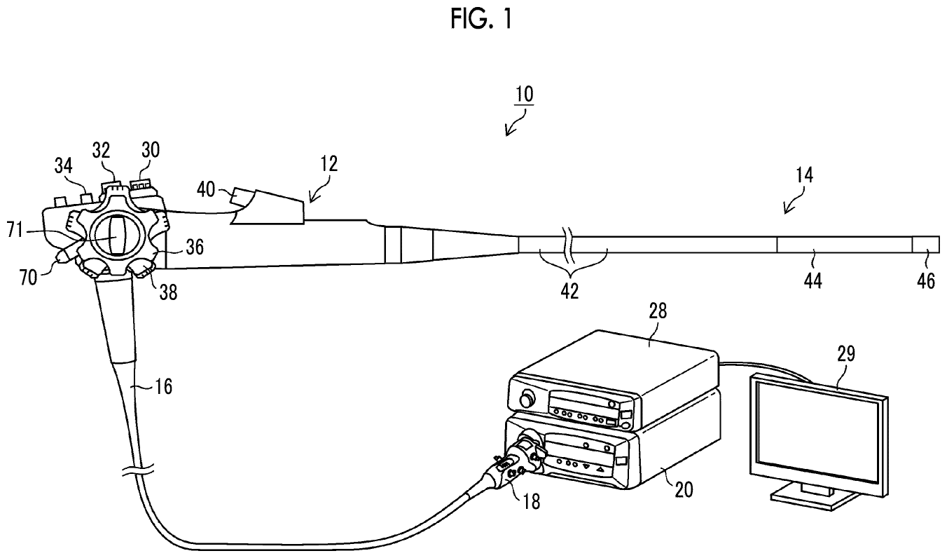

FIG. 1 is an overall configuration view of an endoscope 10 according to the embodiment of the present invention. As shown in FIG. 1, an endoscope 10 includes a hand operating part 12 that is gripped by a practitioner, and an elongated insertion part 14 that is inserted into a body cavity and that has a proximal end part connected to the hand operating part 12.

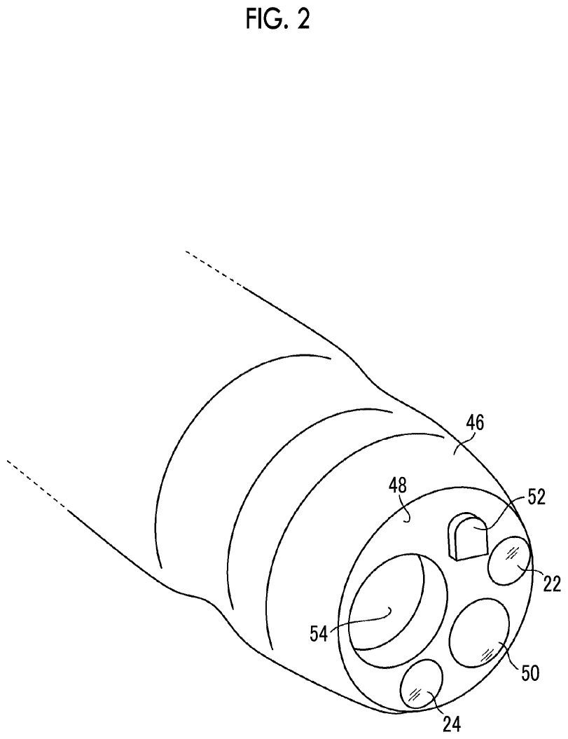

A proximal end part of a universal cable 16 is connected to the hand operating part 12, and a connector 18 is provided at a distal end part of the universal cable 16. The connector 18 is connected to a light source device 20, so that illumination light from the light source device 20 is sent to illumination windows 22 and 24 (with reference to FIG. 2) to be described later. Further, the light source device 20 is electrically connected to a processor unit 28. The connector 18 is electrically connected to the processor unit 28 via the light source device 20. The light source device 20 and the connector 18 can transmit and receive a control signal and an image signal through optical communication. The light source device 20 transmits the control signal and the like, which is transmitted and received by optical communication via the connector 18, to the processor unit 28. The light source device 20 performs wireless power supply for driving the endoscope 10 via the connector 18.

An air and water supply button 30, a suction button 32, and a shutter button 34 operated by a practitioner are installed sequentially in the hand operating part 12, and a pair of operation knobs 36 and 38 are rotatably provided therein. In addition, a forceps insertion part 40 for inserting a treatment tool such as a forceps is provided on a distal end side of the hand operating part 12.

The insertion part 14 is composed of a soft part 42, a bendable part 44, and a distal end hard part 46 from a proximal end part on a hand operating part 12 side toward a distal end part. That is, the insertion part 14 has a distal end hard part 46, a bendable part 44, and a soft part 42 in this order from a distal end side. The bendable part 44 is remotely bent and operated by rotationally operating the operation knobs 36 and 38 provided in the hand operating part 12. In this manner, the distal end hard part 46 can be directed in an up-down direction and a left-right direction. In a case where the operation knob 36 is rotationally operated, the bendable part 44 is bent in the left-right direction. In addition, by rotationally operating the operation knob 38, the bendable part 44 is bent in the up-down direction. The hand operating part 12 is an example of an endoscope operation unit of the embodiment of the present invention. The operation knobs 36 and 38 are examples of a rotation operating part of the embodiment of the present invention.

### Configuration of Distal End Surface

FIG. 2 is an enlarged perspective view of a main part of the distal end hard part 46 as viewed from a distal end side.

An observation window 50, illumination windows 22 and 24, an air and water supply nozzle 52, and a forceps port 54 are provided on a distal end surface 48 of the distal end hard part 46.

An imaging element (not shown) is disposed on a proximal end side of an observation optical system including the observation window 50 in the distal end hard part 46. A signal cable (not shown) is connected to a substrate that supports the imaging element. The signal cable is inserted into the insertion part 14, the hand operating part 12, and the universal cable 16 of FIG. 1 and extends to the connector 18, and is connected to the light source device 20. An electric signal indicating an observation image photoelectrically converted by the imaging element is output from the light source device 20 to the processor unit 28, and then is output to a monitor 29 after the signal is appropriately processed in the processor unit 28. Accordingly, the observation image is displayed on the monitor 29.

An emission end of an optical fiber (not shown) is disposed behind the illumination windows 22 and 24. This optical fiber is inserted into the insertion part 14, the hand operating part 12, and the universal cable 16 of FIG. 1 and extends to the connector 18. Accordingly, in a case where the connector 18 is connected to the light source device 20, illumination light from the light source device 20 is transmitted to the illumination windows 22 and 24 of FIG. 2 via the optical fibers and is emitted forward from the illumination windows 22 and 24.

The air and water supply nozzle 52 communicates with an air and water supply valve (not shown) operated by the air and water supply button 30 of FIG. 1. Accordingly, by operating the air and water supply button 30, air or water can be jetted from the air and water supply nozzle 52 of FIG. 2 toward the observation window 50.

The forceps port 54 communicates with the forceps insertion part 40 via a forceps channel (not shown) inserted into the insertion part 14 of FIG. 1. By inserting various treatment tools such as forceps or a high-frequency scalpel from the forceps insertion part 40, the treatment tools can be led out from the forceps port 54 of FIG. 2. In addition, by operating a suction valve (not shown) by means of the suction button 32, it is possible to suck residue, waste, or the like from the forceps port 54 via the forceps channel.

### Bending Operation Mechanism

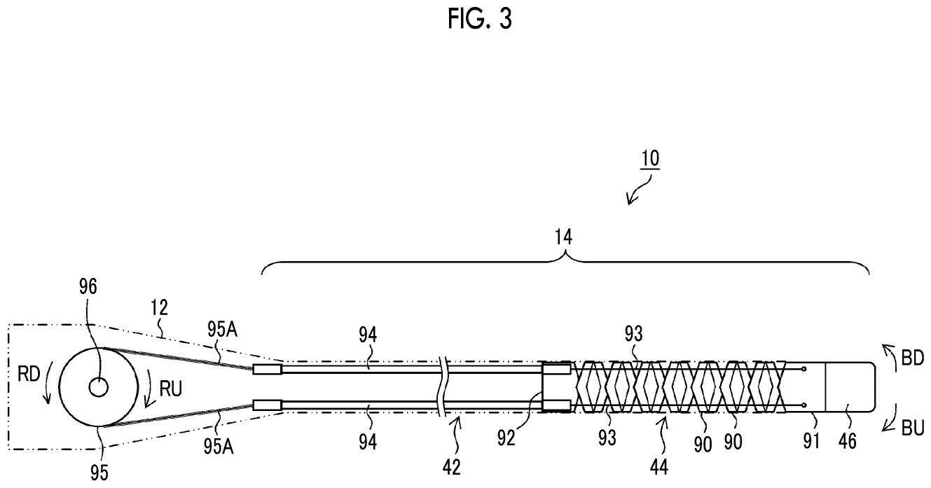

FIG. 3 is a diagram for illustrating a bending operation mechanism for performing a bending operation of the bendable part 44 with the operation knob 38. The bending operation mechanisms corresponding to the operation knobs 36 and 38 each have a common configuration. Here, in order to avoid duplication of description, the bending operation mechanism corresponding to the operation knob 38 will be described as a representative.

As shown in FIG. 3, the bendable part 44 comprises a plurality of nodal rings 90 that are connected in series. The adjacent nodal rings 90 are connected by a connecting pin (not shown). The nodal ring 90 is adapted to be relatively rotatable about an axis of the connecting pin.

Among the nodal rings 90, a distal end ring 91 is fixed to the distal end hard part 46. A proximal end ring 92 is fixed to the soft part 42. Accordingly, the bendable part 44 is configured to be bendable as a whole.

In addition, each distal end part of a pair of operation wires 93 and 93 is fixed inside the distal end ring 91. Each distal end part of a pair of wire guide tubes 94 and 94 is fixed inside the proximal end ring 92. Each operation wire 93 is inserted into each wire guide tube 94.

Each operation wire 93 is disposed inside each nodal ring 90 and is disposed by being inserted into each wire guide tube 94, and the proximal end thereof is fixed to the end part of a chain 95A wound around a sprocket 95 of which the proximal end is disposed inside the hand operating part 12. The sprocket 95 is disposed integrally with the operation knob 38 (not shown) to be rotatable around a rotary shaft 96.

The practitioner rotationally operates the operation knob 38 (with reference to FIG. 1) of the hand operating part 12 to rotate the sprocket 95. In a case where the sprocket 95 is rotated in a direction of an arrow RU, the pair of operation wires 93 and 93 are moved forward and backward in opposite directions, so that the bendable part 44 is bent in a direction of an arrow BU. The operation wire 93 is an example of a bending operation wire of the embodiment of the present invention.

In a case where the sprocket 95 is rotated in a direction of an arrow RD by rotationally operating the operation knob 38, the pair of operation wires 93 and 93 are moved forward and backward in opposite directions, so that the bendable part 44 is bent in a direction of an arrow BD. That is, by rotationally operating the operation knob 38, the bendable part 44 is bent in the up-down direction. The same bending operation mechanism as the operation knob 38 is applied to the operation knob 36, and the bendable part 44 is bent in the left-right direction by rotationally operating the operation knob 36.

### First Embodiment

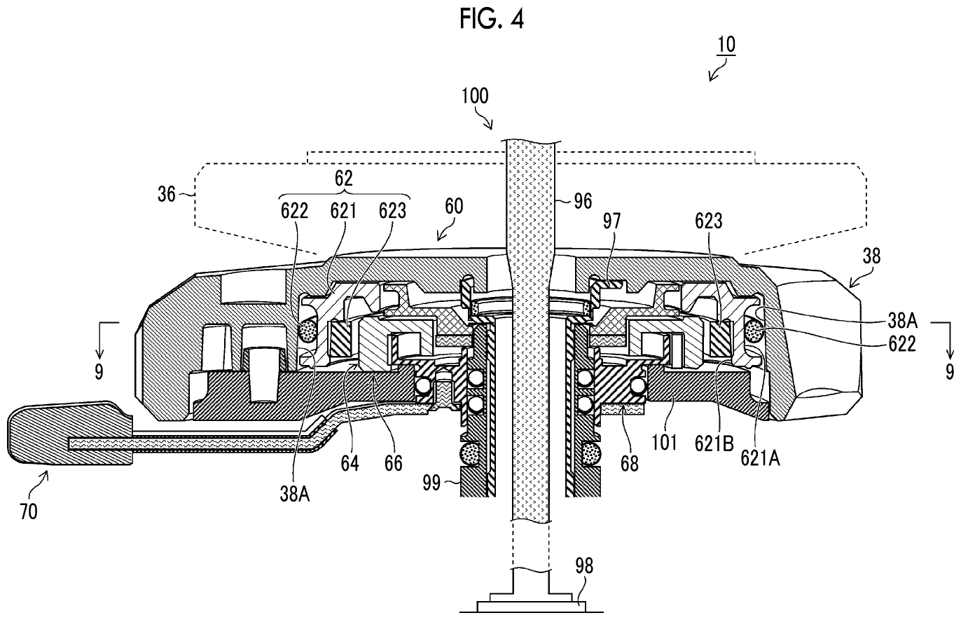

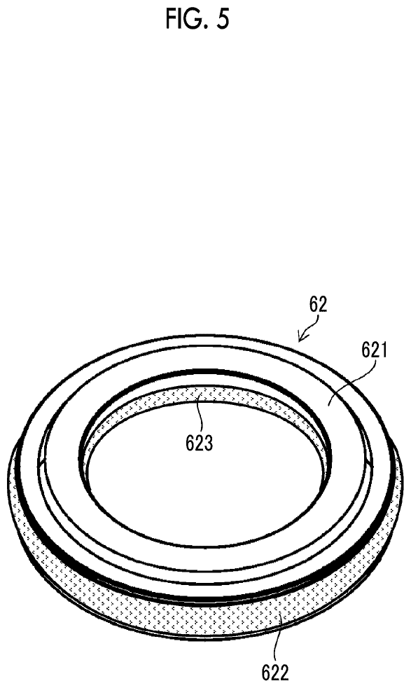

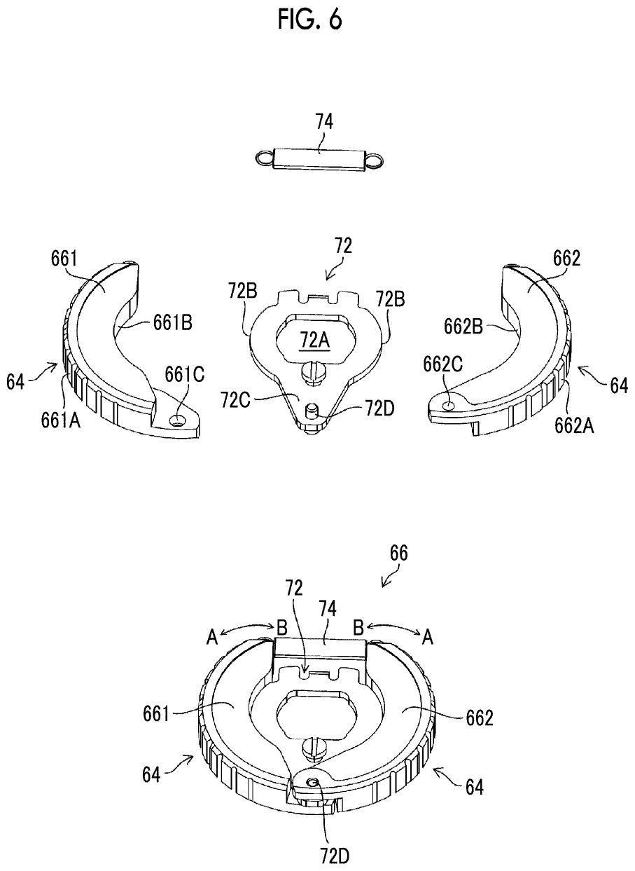

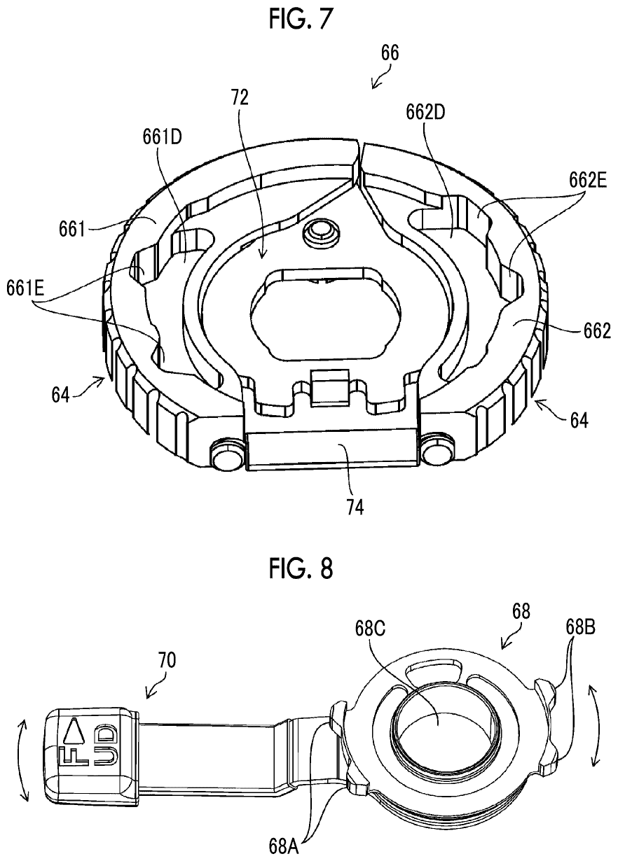

Next, a first embodiment of a brake mechanism 60 that is a feature of the embodiment of the present invention will be described. FIG. 4 is a cross-sectional view of the operation knob 38 taken along a rotary shaft 96. FIG. 5 is a perspective view of an engaging part 62. FIG. 6 is an assembly perspective view of a movable member 66. FIG. 7 is a view of the movable member 66 of FIG. 6 as viewed from an opposite side. FIG. 8 is a perspective view of an operation lever 70 and a rotating body 68.

### Operation Knob Mechanism

First, an operation knob mechanism 100 for performing a bending operation of a bendable part 44 will be described with reference to FIG. 4. As shown in FIG. 4, the operation knob mechanism 100 comprises the rotary shaft 96, a tubular connecting tube 97 that is externally mounted on the rotary shaft 96, and the operation knob 38 that is attached to the connecting tube 97. The rotary shaft 96 is fixed to a support plate 98 at one end. The support plate 98 is fixed to the inside of the hand operating part 12.

The connecting tube 97 is coaxial with the rotary shaft 96 and is externally mounted to be relatively slidable in a circumferential direction. A sprocket 95 (not shown in FIG. 4, with reference to FIG. 3) is attached to the connecting tube 97 at one end side. The sprocket 95 is rotated integrally with the connecting tube 97 around the rotary shaft 96. The connecting tube 97 and the operation knob 38 are mechanically connected to or engaged with each other, and the connecting tube 97 is rotated integrally with the operation knob 38. Therefore, in a case where the operation knob 38 is rotationally operated, this operation force is transmitted to the sprocket 95 through the connecting tube 97. The sprocket 95 rotates according to an operation amount, the pair of operation wires 93 and 93 are moved forward and backward, and the bendable part 44 is bent in the up-down direction. The rotary shaft 96 passes through the operation knob 38 and extends to the operation knob 36. The rotary shaft 96 is an example of a rotary shaft of the embodiment of the present invention.

A housing 99 is externally mounted on an outer side of the connecting tube 97. Further, a cover 101 is attached to the operation knob 38. The cover 101 is rotated around the rotary shaft 96 integrally with the operation knob 38.

### Configuration of Brake Mechanism

Next, the brake mechanism 60 will be described. Here, the brake mechanism 60 is a mechanism that can switch between a free state in which the practitioner can change a bending state of the bendable part 44 by operating the operation knob 38 with his or her fingers and a half-locked state in which the practitioner can maintain a state in which the bending state of the bendable part 44 is changed even in a case where the practitioner releases his or her fingers from the operation knob 38 and can change the bending state of the bendable part 44 in which the bending state is maintained in a case where the practitioner operates the operation knob 38 with his or her fingers.

The brake mechanism 60 includes an engaging part 62 that is frictionally engaged with the operation knob 38, a movable member 66 that has a pressing part 64 which is relatively position-changeable with respect to the engaging part 62, and an operation lever 70 for changing the position of the pressing part 64 of the movable member 66.

As shown in FIGS. 4 and 5, the engaging part 62 is disposed at a position along an inner surface 38A of the operation knob 38. The engaging part 62 comprises a ring member 621, a first elastic member 622, and a second elastic member 623.

The ring member 621 is formed of an annular member centered on the rotary shaft 96. The ring member 621 includes an outer surface 621A that is positioned on an outer peripheral side (a side facing the inner surface 38A of the operation knob 38) of the ring member 621 and an inner surface 621B that is positioned on an inner peripheral side (a side facing the rotary shaft 96) of the ring member 621. The outer surface 621A of the ring member 621 has a shape recessed toward the inner peripheral side (direction of the inner surface 621B) of the ring member 621.

The first elastic member 622 is disposed along the outer peripheral side of the ring member 621. That is, the first elastic member 622 is disposed between the outer surface 621A of the ring member 621 and the inner surface 38A of the operation knob 38. The first elastic member 622 has, for example, an annular shape, and is disposed over the entire surface along the outer surface 621A. The first elastic member 622 is made of rubber or the like. The first elastic member 622 is in contact with the outer surface 621A and the inner surface 38A, and the ring member 621 of the engaging part 62 and the operation knob 38 are frictionally engaged with each other via the first elastic member 622.

The second elastic member 623 is disposed along the inner surface 621B which is an inner peripheral side of the ring member 621. The second elastic member 623 has, for example, an annular shape, and is disposed over the entire surface along the inner surface 621B. The second elastic member 623 is made of rubber or the like.

The engaging part 62 is an example of an engaging part of the embodiment of the present invention. The first elastic member 622 and the second elastic member 623 are examples of a friction material according to the embodiment of the present invention, and are also examples of an elastic member according to the embodiment of the present invention.

In the embodiment, the first elastic member 622 is shown as an example of the friction material, but the configuration is not limited to the configuration shown in the embodiment as long as the ring member 621 and the operation knob 38 of the engaging part 62 can be frictionally engaged with each other, and, for example, a resin or a metal having a rough surface may be used. Even in the case of such friction materials, the ring member 621 of the engaging part 62 and the operation knob 38 are brought into contact with each other so that both the ring member 621 and the operation knob 38 are frictionally engaged with each other. Although the second elastic member 623 is illustrated as a friction material, a resin having a rough surface or a metal may be used as long as the ring member 621 of the engaging part 62 and the pressing part 64 can be frictionally engaged with each other.

As shown in FIGS. 4, 6, and 7, the movable member 66 is disposed further inside the operation knob 38 than the engaging part 62. The movable member 66 includes a first movable member 661 and a second movable member 662. The first movable member 661 comprises an arc-shaped outer surface 661A and an arc-shaped inner surface 661B, and has a crescent shape as a whole. The outer surface 661A comprises a pressing part 64 having an uneven shape along the circumferential direction. A through-hole 661C is formed at one end of the first movable member 661.

Similarly, the second movable member 662 comprises an arc-shaped outer surface 662A and an arc-shaped inner surface 662B and has a crescent shape as a whole. The outer surface 662A comprises a pressing part 64 having an uneven shape along the circumferential direction. A through-hole 662C is formed at one end of the second movable member 662.

The movable member 66 is an example of a movable member of the embodiment of the present invention. In addition, the pressing part 64 that constitutes a part of the first movable member 661 and the second movable member 662 is an example of a pressing part of the embodiment of the present invention.

The first movable member 661 and the second movable member 662 are movably supported by a fixing plate 72. A through-hole 72A through which the rotary shaft 96 and the connecting tube 97 pass is formed in the fixing plate 72. An outer surface 72B of the fixing plate 72 has a shape that follows the inner surface 661B of the first movable member 661 and the inner surface 662B of the second movable member 662. The fixing plate 72 comprises a protruding part 72C on one end side, and a pin 72D parallel to the rotary shaft 96 is provided at a distal end of the protruding part 72C. In addition, the fixing plate 72 is provided not to be rotatable with respect to the rotary shaft 96.

The through-hole 661C of the first movable member 661 and the through-hole 662C of the second movable member 662 are inserted into the pin 72D in opposite directions. The other end of each of the first movable member 661 and the second movable member 662 is connected to a spring 74.

In a case where the other end of each of the first movable member 661 and the second movable member 662 is moved in a direction of an arrow A with the pin 72D as a fulcrum, the movable member 66 is brought into an open state. Further, in a case where the other end of each of the movable members 66 is moved in a direction of an arrow B from the open state, the movable member 66 is brought into a closed state. That is, the movable member 66 is movable in a direction orthogonal to the rotary shaft 96 and can be opened and closed between an open state and a closed state.

The first movable member 661 and the second movable member 662 are biased by the connected spring 74 in the direction of the arrow B in which the other end of each of the first movable member 661 and the second movable member 662 approaches. Therefore, in a free state to be described later, the movable member 66 is in a closed state by a biasing force of the spring 74.

As shown in FIG. 7, a rotating body 68 to be described later is accommodated on the inside of an opposite side surface of the first movable member 661 and the second movable member 662. A first stepped part 661D and a second stepped part 662D are formed on the first movable member 661 and the second movable member 662, respectively. The first stepped part 661D is a dent provided in the first movable member 661, and includes two first engaged parts (engaging recessed parts) 661E extending toward the outer surface 661A. The second stepped part 662D is a dent provided in the second movable member 662, and includes two second engaged parts (engaging recessed parts) 662E extending toward the outer surface 662A.

As shown in FIGS. 4 and 8, the operation lever 70 is composed of a plate-shaped member that protrudes in a direction orthogonal to the rotary shaft 96. The rotating body 68 is connected to the operation lever 70. The rotating body 68 is provided to be rotatable around the rotary shaft 96 on the opposite side surface of the first movable member 661 and the second movable member 662 described above. In a case where the operation lever 70 is rotationally operated (operated to move) in a rotation direction around the rotary shaft 96, the rotating body 68 is rotated around the rotary shaft 96 integrally with the operation lever 70.

A through-hole 68C through which the rotary shaft 96 and the connecting tube 97 pass is formed in the rotating body 68. The rotating body 68 has two first engaging parts (engaging projection parts) 68A that protrude in directions orthogonal to the rotary shaft 96 from an outer peripheral edge thereof, and two second engaging parts (engaging projection parts) 68B that protrude in directions orthogonal to the rotary shaft 96. In a state where the rotating body 68 is accommodated on the opposite side surface of the first movable member 661 and the second movable member 662, the first engaging part 68A is disposed at the first stepped part 661D, and the second engaging part 68B is disposed at the second stepped part 662D.

In a case where the operation lever 70 is rotationally operated, the first engaging part 68A moves in the circumferential direction in the first stepped part 661D, and the second engaging part 68B moves in the circumferential direction in the second stepped part 662D. Accordingly, in a case where the first engaging part 68A and the second engaging part 68B are engaged with the first engaged part 661E and the second engaged part 662E, respectively, the movable member 66 is brought into a closed state by a biasing force of the spring 74. On the other hand, in a case where the first engaging part 68A and the second engaging part 68B are not engaged with the first engaged part 661E and the second engaged part 662E, respectively, the movable member 66 is brought into an open state against the biasing force of the spring 74. That is, the movable member 66 can be opened and closed between the open state and the closed state according to a rotational position of the rotating body 68.

The rotating body 68 is an example of a rotating body of the embodiment of the present invention. The operation lever 70 is an example of a movement operation member according to the embodiment of the present invention.

### Action of Brake Mechanism

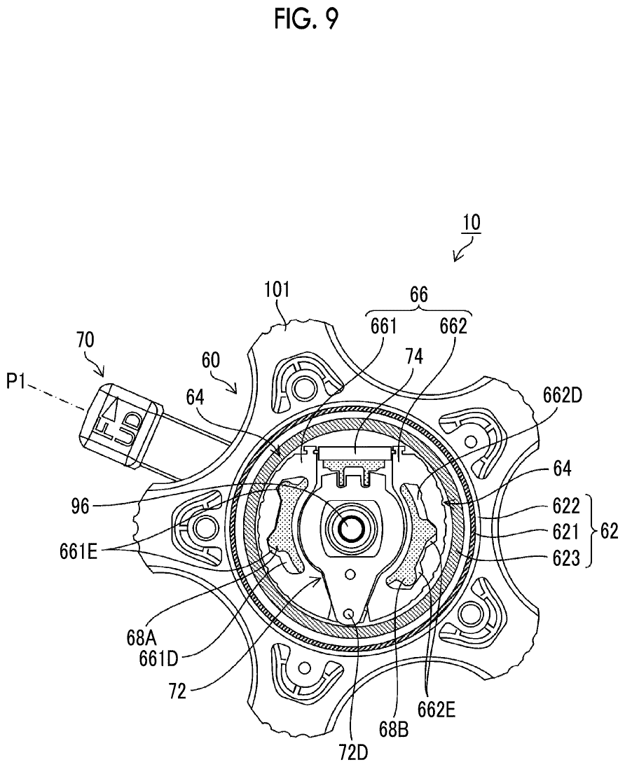

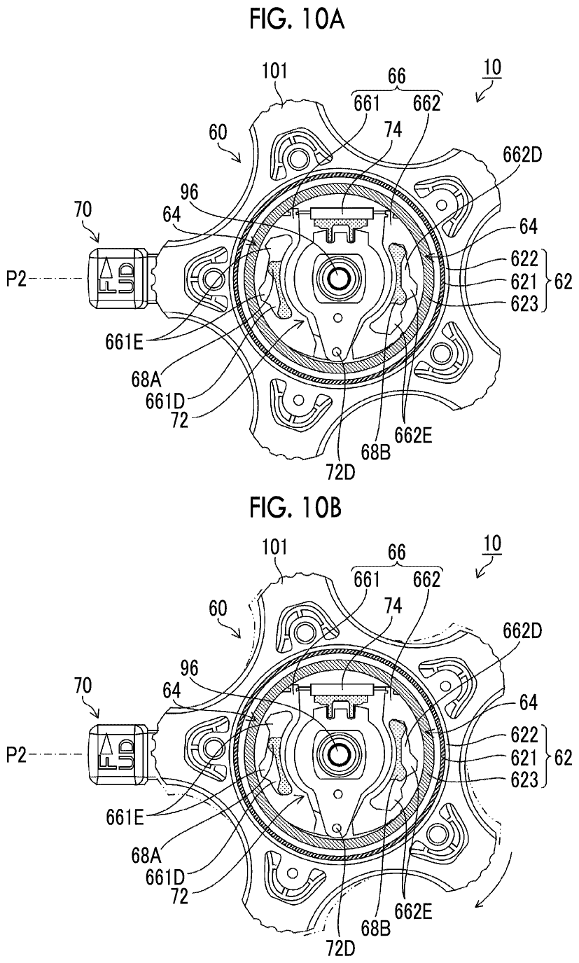

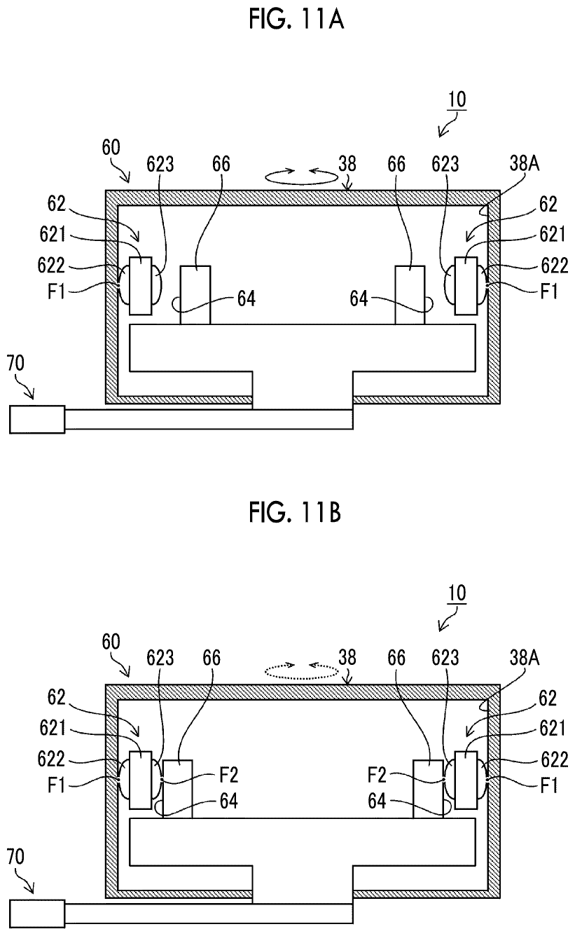

FIG. 9 is a diagram showing a case where the operation knob 38 is in the free state as viewed from the direction of an arrow 9-9 in FIG. 4. FIGS. 10A and 10B are diagrams showing a case where the operation knob 38 is in a half-locked state as viewed from the same direction as in FIG. 9. FIG. 10A shows a state in which the bending state of the bendable part 44 is maintained, and FIG. 10B shows a state in which the operation knob 38 is rotationally operated from the state in FIG. 10A. In FIGS. 9, 10A and 10B, the operation knob 38 is omitted, and the cover 101 that is integrally rotated with the operation knob 38 is displayed in order to facilitate understanding. FIG. 11A is a diagram conceptually showing a case where the operation knob 38 is in the free state. FIG. 11B is a diagram conceptually showing a case where the operation knob 38 is in the half-locked state. The rotating body 68 is omitted in FIGS. 11A and 11B.

In FIG. 9, the operation lever 70 is moved to a first position P1 where the operation knob 38 is in the free state. The rotating body 68 is rotated around the rotary shaft 96 by the operation of moving the operation lever 70, and the rotating body 68 is also moved. At the first position P1 of the operation lever 70, the first engaging part 68A of the rotating body 68 and the first engaged part 661E of the first movable member 661 are in an engaged state. Similarly, the second engaging part 68B of the rotating body 68 and the second engaged part 662E of the second movable member 662 are in an engaged state.

In a case where the first engaging part 68A and the second engaging part 68B are in an engaged state with the first engaged part 661E and the second engaged part 662E, respectively, the biasing force of the spring 74 is dominant with respect to the first movable member 661 and the second movable member 662. The first movable member 661 and the second movable member 662 are moved with the pin 72D as a fulcrum in a direction in which the first movable member 661 and the second movable member 662 approach each other and in a direction orthogonal to the rotary shaft 96, and the movable member 66 is in a closed state. Further, in order to prevent the movable member 66 from being in the open state in a case where the movable member 66 is switched from the half-locked state (FIGS. 10A and 10B) to the free state (FIG. 9), the spring 74 biases the movable member 66 to the closed state by means of the biasing force. As a result, the pressing part 64 of the movable member 66 is positioned at a position spaced apart from the second elastic member 623 of the engaging part 62, that is, the pressing part 64 is positioned at a non-braking position spaced apart from the engaging part 62. The non-braking position of the pressing part 64 is a position of a rotary shaft 96 side (distal side) with respect to the engaging part 62, as compared with a braking position of the pressing part 64 to be described later.

As shown in FIG. 11A, in a case where the pressing part 64 is at the non-braking position, the pressing part 64 does not generate a frictional force with respect to the engaging part 62. However, since the inner surface 38A of the operation knob 38 and the first elastic member 622 of the engaging part 62 are in contact with each other, a frictional force is generated, and the operation knob 38 and the engaging part 62 are frictionally engaged with each other by the frictional force (first frictional force F1). Since the pressing part 64 does not generate a frictional force with respect to the engaging part 62, the operation knob 38 is in a free state of rotating integrally with the engaging part 62. The direction of the bendable part 44 can be changed by rotationally operating the operation knob 38. Meanwhile, in a case where the fingers are released from the operation knob 38, the bendable part 44 tends to return to the state before the change.

In FIG. 10A, the operation lever 70 is moved to a second position P2 where the operation knob 38 is in the half-locked state. The rotating body 68 is rotated around the rotary shaft 96 by the operation of moving the operation lever 70, and the rotating body 68 is also moved. At the second position P2 of the operation lever 70, the first engaging part 68A and the first engaged part 661E are in a disengaged state. Similarly, the second engaging part 68B and the second engaged part 662E are in a disengaged state.

In a case where the first engaging part 68A and the second engaging part 68B are in a disengaged state with the first engaged part 661E and the second engaged part 662E, respectively, the first engaging part 68A pushes an inner wall of the first stepped part 661D in the direction orthogonal to the rotary shaft 96. In addition, the second engaging part 68B pushes an inner wall of the second stepped part 662D in the direction orthogonal to the rotary shaft 96. The first movable member 661 and the second movable member 662 move in a direction in which the first movable member 661 and the second movable member 662 are separated from each other and in a direction orthogonal to the rotary shaft 96 with the pin 72D as a fulcrum against the biasing force of the spring 74, and the movable member 66 is in an open state. As a result, the pressing part 64 of the movable member 66 is positioned at a position coming into contact with the second elastic member 623 of the engaging part 62, that is, the pressing part 64 is positioned at the braking position in contact with the engaging part 62.

As shown in FIG. 11B, in a case where the pressing part 64 is at the braking position, the pressing part 64 is in contact with the second elastic member 623 of the engaging part 62, so that a frictional force (second frictional force F2) is generated, and the engaging part 62 and the pressing part 64 are frictionally engaged with each other by the frictional force. The pressing part 64 is not rotatable with respect to the rotary shaft 96, and the rotation of the engaging part 62 is restricted by the second frictional force F2 by the pressing part 64 coming into contact with the engaging part 62, and further, the rotation of the operation knob 38 is restricted by a load resistance (the first frictional force F1). Therefore, in a case where the practitioner releases his or her fingers from the operation knob 38, the operation knob 38 is restricted from rotating. Therefore, the practitioner can maintain the state in which the bending state of the bendable part 44 is changed.

In FIG. 10B, as in FIG. 10A, the operation lever 70 is moved to the second position P2 where the operation knob 38 is in the half-locked state. Since the operation knob 38 and the engaging part 62 are configured to be relatively movable, as shown in FIG. 10B, the practitioner can change the bending state of the bendable part 44 without releasing the half-locked state of the operation knob 38 by rotationally operating the operation knob 38.

Specifically, as shown in FIG. 11B, in a case where the pressing part 64 is at the braking position and the operation knob 38 is in the half-locked state, a first frictional force F1 between the operation knob 38 and the engaging part 62 and a second frictional force F2 between the engaging part 62 and the pressing part 64 are generated. The engaging part 62 is in a state of not rotating integrally with the operation knob 38 because the rotation of the engaging part 62 is restricted by the second frictional force F2. Therefore, in a case where the operation knob 38 is rotationally operated, the operation knob 38 is relatively rotated against the load resistance (first frictional force F1) by the engaging part 62, the connecting tube 97 connected to the operation knob 38 is rotated to rotate the sprocket 95, and the bending state of the bendable part 44 is changed. With this configuration, it is possible to maintain the bending state of the bendable part 44 and to generate an appropriately changeable braking force of the bending state of the bendable part 44 on the operation knob 38. In this case, it is preferable that the first frictional force F1 is smaller than the second frictional force F2.

In the present embodiment, in a case where the operation knob 38 is in the half-locked state, the load resistance of the operation knob 38 is a magnitude of the first frictional force F1. The magnitude of the first frictional force F1 can be determined in advance based on a positional relationship between the operation knob 38 and the engaging part 62. That is, the operation knob 38 can be set to a size that allows the operation knob 38 to be rotationally moved by the operation of the fingers. Further, since there are few related components or the like that generate the first frictional force F1, variations in the magnitude of the first frictional force F1 can be suppressed. In a case where the operation knob 38 is in the half-locked state, the magnitude of the first frictional force F1 does not vary. Therefore, it is possible to realize stable operability by suppressing variations in the bending operation of the bendable part 44 according to the operation knob 38. Further, even in a case where the operation knob 38 is in a free state, since the operation knob 38 and the engaging part 62 are integrated, the operability is stable.

In addition, as shown in FIGS. 9 and 10, the movable member 66 is configured to be movable in a direction orthogonal to the rotary shaft 96 of the operation knob 38. According to this configuration, in a case where the movable member 66 is accommodated in the operation knob 38, the operation knob 38 can be made thinner in a direction of the rotary shaft 96 and can be made larger in a direction orthogonal to the rotary shaft 96. Therefore, the operation knob 38 can be brought close to the fingers, and the operability of the operation knob 38 can be improved.

Further, as shown in FIGS. 9 and 11, in a case where the pressing part 64 is positioned at the non-braking position, the pressing part 64 is positioned at a position spaced apart from the engaging part 62. Therefore, in a case where the operation knob 38 is in the free state, it is possible to suppress the occurrence of a load resistance on the operation knob 38 and to improve the operability of the operation knob 38.

In the present embodiment, as one of the preferred aspects, the configuration has been described in which the pressing part 64 is positioned at a non-braking position and the pressing part 64 is positioned at a position spaced apart from the engaging part 62. However, the embodiment of the present invention is not limited to this, and the pressing part 64 may not necessarily be positioned at a position spaced apart from the engaging part 62. That is, the non-braking position of the pressing part 64 may be a position on the rotary shaft 96 side (distal side) with respect to the engaging part 62, as compared with the braking position. Also in this case, it is possible to obtain the same effect as in the present embodiment.

Although the brake mechanism 60 of the operation knob 38 has been described, the brake mechanism 60 of the embodiment can be applied to the operation knob 36. An operation knob 71 (with reference to FIG. 1) is suitably applied to the operation knob 36 instead of the operation lever 70. The operation knob 71 is an example of a movement operation member according to the embodiment of the present invention.

### Second Embodiment

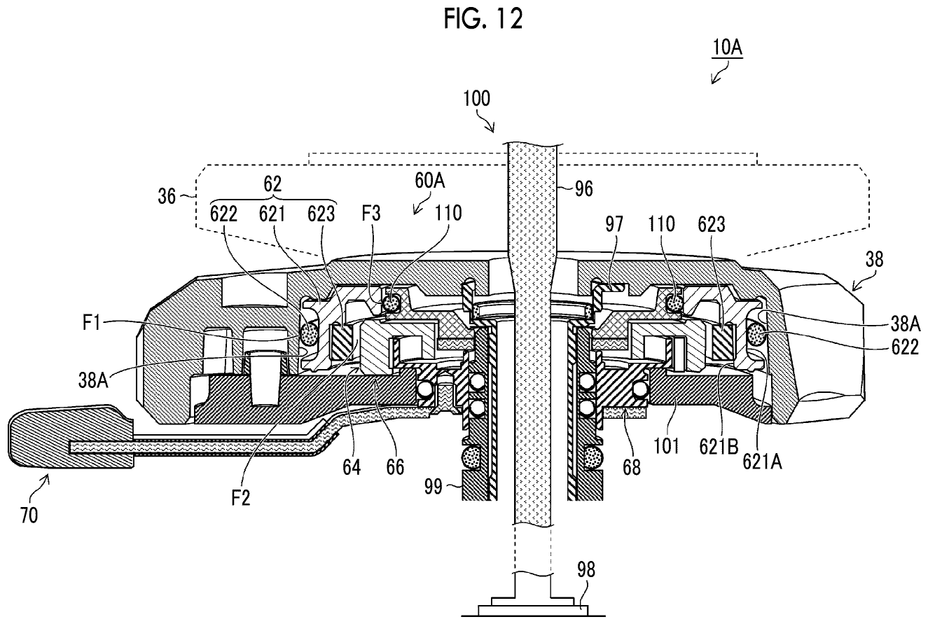

FIG. 12 is a diagram for explaining a second embodiment. In FIG. 12, the same parts as those in the above-described first embodiment are designated by the same reference numerals, and the description thereof will not be repeated.

A brake mechanism 60A of an endoscope 10A of the second embodiment shown in FIG. 12 is different from the endoscope 10 of the first embodiment in that the brake mechanism 60A comprises an O-ring 110 that abuts on the ring member 621 of the engaging part 62.

In a free state in which the pressing part 64 is positioned at the non-braking position, the O-ring 110 is engaged with the engaging part 62 via frictional engagement (third frictional force F3). A third frictional force F3 between the O-ring 110 and the engaging part 62 serves as a load resistance with respect to the operation knob 38. According to the O-ring 110 disposed as described above, the operation knob 38 can be slowly rotated and moved in a case where a hand is released from the operation knob 38. In order to integrally rotate the operation knob 38 and the engaging part 62 at the first frictional force F1, it is preferable that the third frictional force F3 is smaller than the first frictional force F1.

In a half-locked state in which the pressing part 64 is positioned at the braking position, the rotation of the engaging part 62 is restricted by the second frictional force F2 from the pressing part 64 and by the third frictional force F3 from the O-ring 110. Similarly to the first embodiment, the operation knob 38 can maintain a state in which the bendable part 44 is changed in a bending state in a case where the practitioner releases his or her fingers from the operation knob 38 because the operation knob 38 is restricted from rotating by the first frictional force F1 from the engaging part 62 and by the second frictional force F2 from the pressing part 64. In FIG. 12, the pressing part 64 and the engaging part 62 are not in contact with each other. However, a second frictional force F2 generated in a case where the pressing part 64 and the engaging part 62 are in contact with each other is illustrated for convenience.

In addition, in the half-locked state, in a case where the operation knob 38 is rotationally operated, the engaging part 62 is restricted from rotating integrally with the operation knob 38 due to the second frictional force F2 and the third frictional force F3, and the operation knob 38 is relatively rotatable against the load resistance (first frictional force F1) set by the engaging part 62. Accordingly, a bending state of the bendable part 44 can be changed.

Also in the second embodiment provided with the O-ring 110, in the half-locked state, the load resistance of the operation knob 38 is the first frictional force F1 from the engaging part 62. As described above, since the first frictional force F1 can be set in advance and variations can be suppressed, variations in the bending operation of the bendable part 44 by the operation knob 38 can be suppressed, and stable operability can be realized.

Although the O-ring 110 is illustrated as a member that generates the third frictional force F3 in the engaging part 62, the structure, materials, and the like are not limited as long as the third frictional force F3 can be generated.

### Third Embodiment

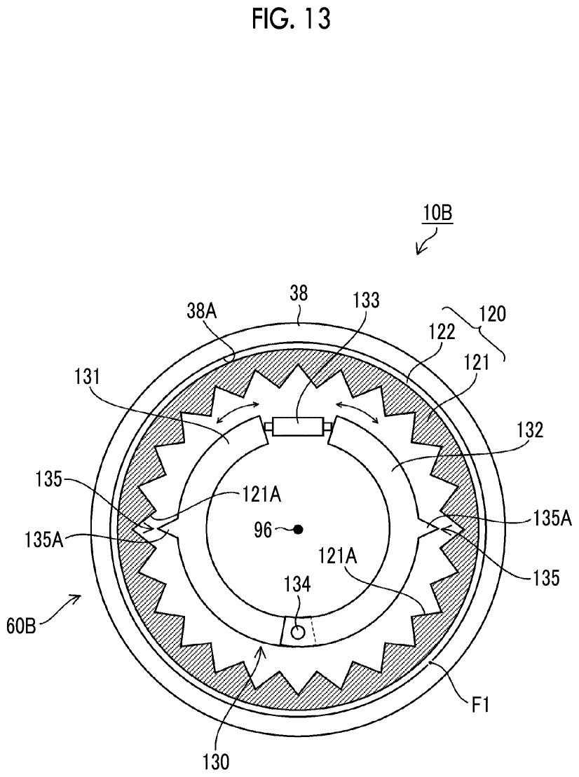

FIG. 13 is a diagram for explaining a third embodiment. In FIG. 13, the same parts as those in the above-described first embodiment are designated by the same reference numerals, and the description thereof will not be repeated.

A brake mechanism 60B of an endoscope 10B of the third embodiment shown in FIG. 13 has a structure of an engaging part 62, a pressing part 64, and a movable member 66 different from the endoscope 10 of the first embodiment.

As shown in FIG. 13, an engaging part 120 comprises a ring member 121 and an elastic member 122 that is disposed on an outer peripheral side of the ring member 121 (a side facing the inner surface 38A of the operation knob 38). The elastic member 122 generates the first frictional force F1 in advance with the operation knob 38, similarly to the first elastic member 622 of the first embodiment.

The ring member 121 comprises a plurality of recessed parts 121A on an inner surface of an inner peripheral side (a side facing a movable member 130) of the ring member 121 in the circumferential direction. The recessed part 121A is not particularly limited as long as the recessed part 121A has a cross-sectional shape that can engage with a projection part 135A of a pressing part 135 to be described later, but is configured as a triangular shape that gradually tapers toward a bottom portion of the recessed part 121A as an example.

The movable member 130 is formed of a first movable member 131 and a second movable member 132. The first movable member 131 and the second movable member 132 have the shape of an arch as viewed from the direction of the rotary shaft 96. One end of each of the first movable member 131 and the second movable member 132 is fixed by a pin 134. The other end of each of the first movable member 131 and the second movable member 132 is biased to get close to one another by, for example, springs 133. The first movable member 131 and the second movable member 132 are movable with the pin 134 as a fulcrum, and the movable member 130 is configured to be openable and closable between the open state and the closed state.

Two projection parts 135A are provided on the pressing part 135 (an outer surface of each of the first movable member 131 and the second movable member 132) of the movable member 130. The projection part 135A has a cross-sectional shape that can be engaged with the recessed part 121A of the above-described ring member 121, and is formed in, for example, a triangular shape that gradually tapers toward the distal end of the projection part 135A.

In a case where the movable member 130 is in the closed state, the pressing part 135 is positioned at the non-braking position, and the operation knob 38 is in a free state. In this case, by rotationally operating the operation knob 38, the operation knob 38 and the engaging part 120 are integrally rotated by the first frictional force F1, and the direction of the bendable part 44 can be changed.

In a case where the movable member 130 is in the open state, the pressing part 135 is positioned at the braking position where the pressing part 135 comes into contact with the engaging part 120 and is in the half-locked state. In the half-locked state, the projection part 135A of the pressing part 135 is engaged with the recessed part 121A of the engaging part 120, and the rotation of the engaging part 120 is restricted. In this case, the rotation operation of the operation knob 38 is restricted by the first frictional force F1 between the engaging part 120 and the operation knob 38. That is, with the uneven engagement between the projection part 135A and the recessed part 121A and the first frictional force F1, as in the first embodiment, the bending state of the bendable part 44 is maintained even in a case where the practitioner releases his or her fingers from the operation knob 38.

In addition, in the half-locked state, since the projection part 135A of the pressing part 135 and the recessed part 121A of the engaging part 120 are unevenly engaged with each other, and the engaging part 120 is restricted from rotating integrally with the operation knob 38, the operation knob 38 is relatively rotatable against the load resistance (first frictional force F1) set by the engaging part 120.

In the third embodiment, since the engaging part 120 and the pressing part 135 are unevenly engaged with each other, the rotation of the engaging part 120 can be restricted more reliably.

The projection part 135A is an example of a projection part of the embodiment of the present invention. The recessed part 121A is an example of a recessed part of the embodiment of the present invention. As long as the projection part and the recessed part can be unevenly engaged with each other, the shape thereof is not particularly limited, and may be a rectangular shape.

### Fourth Embodiment

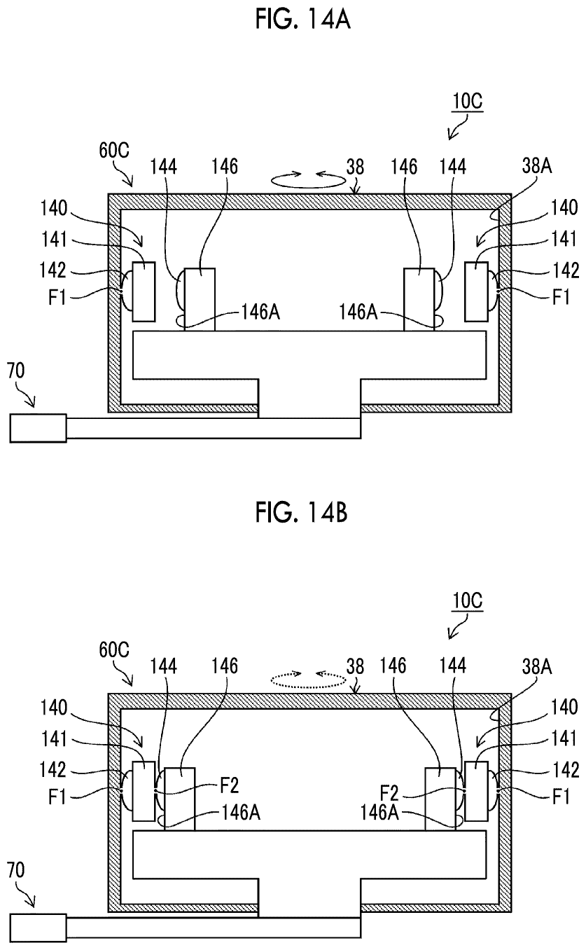

FIGS. 14A and 14B are diagrams for explaining a fourth embodiment. In FIGS. 14A and 14B, the same parts as those in the above-described present embodiment are designated by the same reference numerals, and description thereof will not be repeated. FIG. 14A is a diagram conceptually showing a case where the operation knob 38 is in a free state. FIG. 14B is a diagram conceptually showing a case where the operation knob 38 is in a half-locked state. The rotating body 68 is not shown in FIGS. 14A and 14B.

A brake mechanism 60C of an endoscope 10C of the fourth embodiment shown in FIGS. 14A and 14B has a structure of an engaging part 62, a pressing part 64, and a movable member 66 different from the endoscope 10 of the first embodiment.

As shown in FIG. 14A, an engaging part 140 includes a ring member 141 and an elastic member 142 that is disposed on an outer periphery side (a side facing the inner surface 38A of the operation knob 38) of the ring member 141. Similarly to the first elastic member 622 of the first embodiment, the elastic member 142 can generate the first frictional force F1 between the elastic member 142 and the operation knob 38 in advance.

A pressing part 144 is attached to an outer surface 146A side (engaging part 140 side) of a movable member 146. The pressing part 144 is formed of, for example, the same elastic member as the second elastic member 623 of the first embodiment. The pressing part 144 functions as a friction material for frictionally engaging with the engaging part 140. In addition, the movable member 146 can have the same configuration as the movable member 66 of the first embodiment.

In the free state in which the pressing part 144 of FIG. 14A is positioned at the non-braking position, the operation knob 38 and the engaging part 140 are integrally rotated by the first frictional force F1. The direction of the bendable part 44 can be changed by rotationally operating the operation knob 38.

In the half-locked state in which the pressing part 144 of FIG. 14B is positioned at the braking position, the pressing part 144 is brought into contact with the engaging part 140, and the second frictional force F2 is generated between the pressing part 144 and the engaging part 140. Similarly to the first embodiment, the bending state of the bendable part 44 is maintained in a case where the practitioner releases his or her fingers from the operation knob 38 due to the second frictional force F2 and the first frictional force F1.

In addition, in a case where the operation knob 38 is rotated, the engaging part 62 is restricted from rotating integrally with the operation knob 38 by the second frictional force F2, and the operation knob 38 is relatively rotatable with respect to the engaging part 62 against the load resistance (the first frictional force F1) set by the engaging part 62.

In the fourth embodiment, since the pressing part 144 is configured with the elastic member and functions as the friction material, it is possible to obtain the same effect as in the first embodiment even in a case where the elastic member is not provided on the engaging part 140 side.

The endoscope according to the embodiment of the present invention has been described above in detail, but the present invention may include some improvements or modifications without departing from the scope of the present invention.

### EXPLANATION OF REFERENCES

10: endoscope
10A: endoscope
10B: endoscope
10C: endoscope
12: hand operating part
14: insertion part
16: universal cable
18: connector
20: light source device
22: illumination window
24: illumination window
28: processor unit
29: monitor
30: air and water supply button
32: suction button
34: shutter button
36: operation knob
38: operation knob
38A: inner surface
40: forceps insertion part
42: soft part
44: bendable part
46: distal end hard part
48: distal end surface
50: observation window
52: air and water supply nozzle
54: forceps port
60: brake mechanism
60A: brake mechanism
60B: brake mechanism
60C: brake mechanism
62: engaging part
621: ring member
621A: outer surface
621B: inner surface
622: first elastic member
623: second elastic member
64: pressing part
66: movable member
661: first movable member
661A: outer surface
661B: inner surface
661C: through-hole
661D: first stepped part
661E: first engaged part
662: second movable member
662A: outer surface
662B: inner surface
662C: through-hole
662D: second stepped part
662E: second engaged part
68: rotating body
68A: first engaging part
68B: second engaging part
68C: through-hole
70: operation lever
71: operation knob
72: fixing plate
72A: through-hole
72B: outer surface
72C: protruding part
72D: pin
74: spring
90: nodal ring
91: distal end ring
92: proximal end ring
93: operation wire
94: wire guide tube
95: sprocket
95A: chain
96: rotary shaft
97: connecting tube
98: support plate
99: housing
100: operation knob mechanism
101: cover
110: O-ring
120: engaging part
121: ring member
121A: recessed part
122: elastic member
130: movable member
131: first movable member
132: second movable member
133: spring
134: pin
135: pressing part
135A: projection part
140: engaging part
141: ring member
142: elastic member
144: pressing part
146: movable member
146A: outer surface
A: arrow
B: arrow
BD: arrow
BU: arrow
RD: arrow
RU: arrow
F1: first frictional force
F2: second frictional force
F3: third frictional force

What is claimed is:

1. An endoscope comprising:
a rotation operating part that is rotatably provided on an endoscope operation unit and that moves a bending operation wire forward and backward by being rotationally operated;
an engaging part that frictionally engages with the rotation operating part; and
a pressing part that is movable between a braking position coming into contact with the engaging part and a non-braking position positioned on a distal side with respect to the engaging part from the braking position,
wherein the rotation operating part and the engaging part are integrally rotatable in a case where the pressing part is positioned at the non-braking position, and the rotation operating part and the engaging part are relatively rotatable in a case where the pressing part is positioned at the braking position.

2. The endoscope according to claim 1,
wherein the pressing part is configured to be movable in a direction orthogonal to a rotary shaft of the rotation operating part.

3. The endoscope according to claim 1,
wherein the non-braking position is a position at which the pressing part is separated from the engaging part.

4. The endoscope according to claim 1,
wherein the engaging part and the rotation operating part are frictionally engaged with each other by a friction material.

5. The endoscope according to claim 4,
wherein the friction material is made of an elastic member.

6. The endoscope according to claim 1, further comprising:
a movement operation member that operates to move the pressing part between the non-braking position and the braking position.

7. The endoscope according to claim 6, further comprising:
a movable member that moves the pressing part between the non-braking position and the braking position in a case where the movement operation member is operated to move.

8. The endoscope according to claim 7, further comprising:
a rotating body that is rotatable around a rotary shaft of the rotation operating part in a case where the movement operation member is operated to move,
wherein the movable member is configured to be opened and closed between an open state and a closed state according to a rotational position of the rotating body, and
the pressing part is positioned at the non-braking position in a case where the movable member is in the closed state, and the pressing part is positioned at the braking position in a case where the movable member is in the open state.

9. The endoscope according to claim 1,
wherein the pressing part and the engaging part are frictionally engaged with each other in a case where the pressing part is positioned at the braking position.

10. The endoscope according to claim 9,
wherein the pressing part and the engaging part are frictionally engaged with each other by a friction material.

11. The endoscope according to claim 9,
wherein in a case where the pressing part is positioned at the braking position, a frictional force generated between the engaging part and the rotation operating part is defined as a first frictional force, and a frictional force generated between the pressing part and the engaging part is defined as a second frictional force, the first frictional force is smaller than the second frictional force.

12. The endoscope according to claim 1,
wherein the pressing part and the engaging part are unevenly engaged with each other in a case where the pressing part is positioned at the braking position.

13. The endoscope according to claim 12,
wherein the pressing part is provided with a projection part, and the engaging part is provided with a recessed part that engages with the projection part.
